(19) United States
(12) Patent Application Publication
(10) Pub. No.: US 2021/0183524 A1
(43) Pub. Date: Jun. 17, 2021

(54) METHOD AND SYSTEM FOR PROVIDING INTERPRETATION INFORMATION ON PATHOMICS DATA

(71) Applicant: Lunit, Inc., Seoul (KR)

(72) Inventor: Jeong Hoon LEE, Seoul (KR)

(21) Appl. No.: 16/832,142

(22) Filed: Mar. 27, 2020

(30) Foreign Application Priority Data
Dec. 16, 2019 (KR) 10-2019-0168111

(51) Int. Cl. G16H 50/70; G16B 50/10; G16H 10/40; G16H 30/20; G16H 30/40; G16H 70/60

(52) U.S. Cl. CPC G16H 50/70; G16B 50/10; G16H 70/60; G16H 30/20; G16H 30/40; G16H 10/40

(57) ABSTRACT

An operation method of a computing device operated by at least one processor is provided. The operation method comprises receiving pathomics data samples analyzed from slide images of patients and gene samples of the patients, generating a plurality of gene modules by grouping genetic information included in the gene samples, annotating information of databases significantly enriched in each of the gene modules, to a corresponding gene module, based on one-to-one correlation values between the plurality of the gene modules and a plurality of individual pathomics data representing the pathomics data samples, extracting connectivity between the plurality of the individual pathomics data and the plurality of gene modules, and connecting information annotated to each gene module and the individual pathomics data connected to the corresponding gene module.

### CROSS-REFERENCES TO THE RELATED APPLICATIONS

This application claims priority from Korean Patent Application No. 10-2019-0168111 filed on Dec. 16, 2019, in the Korean Intellectual Property Office, the disclosure of which is incorporated herein by reference in its entirety.

### BACKGROUND

### (a) Field

The present disclosure relates to digital pathology.

### (b) Description of the Related Art

Researches to figure out whether a patient suffers a disease or to determine a status of the disease are have been performed through various molecular markers such as an mRNA, a protein, and the like. Recently, in order to find a biomarker that enables to figure out the disease status more accurately and consistently, researches for finding a molecular marker showing a specific pattern have been performed by using various omics data for each disease status.

Meanwhile, pathology is the study of organic and functional changes in the tissues and organs of the body where inflicted by a disease. In methodological aspect, pathology is rapidly shifting from traditional pathology where tissues or cells taken from a human body are placed on a glass slide and observed with an optical microscope, to digital pathology.

Digital pathology refers to a system that converts the glass slide into a digital image, and analyzes, stores, and manages the digital images. As a method for converting the glass slide into a digital image, a whole slide imaging (WSI) method may be used, in which part or all of the contents of the glass slide is scanned with high magnification and then digitized.

A slide image obtained through WSI provides a large amount of visual information that can be seen at the cell level, and thus may be used as important data for diagnostic medicine. A recently developed AI pathology analyzer such as Lunit SCOPE enables comprehensive analysis of tissue cells and further enables a large amount of data not having been utilized so far to be made in a feasible form. In particular, the Lunit SCOPE may generate data called “pathomics” from the slide image, through cell classification, tissue classification, and structure classification. The term “pathomics” refers to histopathological data containing information of all histologic components obtained from a pathology slide image. Features extracted from the slide image through histopathologic analysis may be used as a biomarker for prognostic prediction, reactivity prediction of anticancer drugs, and clinical decision.

On the other hand, although the pathomics data contains a lot of information, biological and/or medical explanation and interpretation of the histological data should comes first in order to clinically utilize such information. However, histopathology techniques up to now does not biologically and/or medically interpret the extracted result (histopathology data) from the slide image, and not provide the biological and medical meaning thereof. Thus, it is difficult for a user to understand the features extracted from the AI slide image analyzer. Additionally, due to the absence of biological and medical information of the features extracted from the slide image, there is a limit that the means for evaluating the reliability of the AI pathology analyzer is not provided.

### SUMMARY

The present disclosure provides a method and a system for providing biological and/or medical interpretation information of pathomics data extracted from a slide image.

The present disclosure provides a method and a system for analyzing relationship between pathomics data and modularized genetic information, and providing biological and/or medical interpretation information of pathomics data by using a function of a gene module related to the pathomics data.

The present disclosure provides a method and a system for visualizing biological and/or medical interpretation information of pathomics data.

According to an embodiment of the present disclosure, an operation method of a computing device operated by at least one processor may be provided. The operation method comprises receiving pathomics data samples analyzed from slide images of patients and gene samples of the patients, generating a plurality of gene modules by grouping genetic information included in the gene samples, annotating information of databases significantly enriched in each of the gene modules, to a corresponding gene module, based on one-to-one correlation values between the plurality of the gene modules and a plurality of individual pathomics data representing the pathomics data samples, extracting connectivity between the plurality of the individual pathomics data and the plurality of gene modules, and connecting information annotated to each gene module and the individual pathomics data connected to the corresponding gene module.

Generating the plurality of gene modules may comprises, based on correlations among RNAs and/or proteins included in the gene samples, modularizing the RNAs and/or proteins into the plurality of gene modules.

Each of the gene samples may include quantitative data that are obtained through measuring the RNAs and/or proteins by transcriptome analysis and/or proteome analysis.

The databases may be selected from databases that provide relationship information between biologically discovered genes and functions, gene feature information including pathways and interaction information, and medicine and pharmacy information.

Annotating information of databases may comprise determining information of the databases significantly enriched in each of the gene modules through enrichment analysis.

Extracting the connectivity may comprise shortening a value of each of the gene modules in a designated method and determining existence of a relationship between each of the gene modules and each individual pathomics data by using the shortened value of each of the gene modules.

The operation method may further comprises providing information annotated to each of the gene modules as interpretation information of individual pathomics data connected to corresponding gene module.

The individual pathomics data may be a parameter representing cellular information and structural information of a pathological image, and a value of the individual pathomics data may be determined by a representative value of the quantitative data of corresponding parameter in the pathomics data samples.

According to an embodiment, a computing device may be provided. The computing device may comprise a memory and at least one processor that executes instructions of a program loaded in the memory. The processor may generates a plurality of gene modules by grouping genetic information of patients, determine a gene module correlated with pathomics data among the plurality of gene modules, and connect information of databases significantly enriched in each of the gene modules to the pathomics data correlated with corresponding gene module. The pathomics data may be composed of parameters representing cellular information and structural information of pathological images and each parameter may be represented as quantitative data. The pathological images may be obtained from the patients who provide the genetic information.

The processor may modularize RNAs and/or proteins into the plurality of gene modules, based on correlations among the RNAs and/or the proteins included in the genetic information.

The processor may determine information of the databases significantly enriched in each genetic module through enrichment analysis.

The processor may shorten a value of each of the gene modules in a designated method, calculate a correlation value between each of the gene module and individual pathomics data included in the pathomics data by using the shortened value of each gene module, and make a relationship between the individual pathomics data and a gene module whose correlation value is equal to or greater than a threshold.

The processor may annotate information of databases significantly enriched in each of the gene modules to a corresponding gene module, and provide the information annotated to each of the gene modules as interpretation information of pathomics data connected to corresponding gene module.

According to an embodiment, a program stored on a non-transitory computer-readable storage medium may be provided. The program may comprise instructions for causing a computing device to execute generating a plurality of gene modules by grouping genetic information of patients, annotating information of databases significantly enriched in each gene module to a corresponding gene module, determining a gene module correlated with pathomis data based on correlation values between the pathomics data and the plurality of genetic modules, and storing connectivity between the plurality of the gene modules and the pathomics data extracted based on the correlation values, and the information annotated to each of the gene modules. The pathomics data may be composed of parameters representing cellular information and structural information of pathological images, and each of the parameters may be represented as quantitative data. The pathological images may be information obtained from the patients who provide the genetic information.

Annotating the information of databases may comprise determining information of the databases significantly enriched in each of the gene modules through enrichment analysis, and annotating the information of the databases significantly enriched in each of the gene modules to a corresponding gene module.

The program may further comprises instructions for causing a computing device to execute providing the information annotated to each of the gene modules as interpretation information of the pathomics data based on a connectivity between the pathomics data and the plurality of gene modules.

According to some embodiments, by providing interpretation information on pathomics data extracted from slide images, biological meaning and medical meaning of the pathomics data may be interpreted and inferred.

According to some embodiments, the utilization of pathomics data applicable to biological and/or medical interpretation may be improved, and interpretation of features extracted from slide images may contribute to discovery of a biomarker for prognostic prediction, reactivity prediction of anticancer drugs, and clinical decision.

According to some embodiments, a proof for reliability of performance of an AI pathology analyzer may be afforded by providing pathomics data and biological and/or medical information connected thereto.

### BRIEF DESCRIPTION OF THE DRAWINGS

FIG. 1 is a diagram for explaining an AI pathology analyzer according to an embodiment.

FIG. 2 is a block diagram illustrating a system for providing interpretation information of pathomics data according to an embodiment.

FIG. 3 is an example of a relationship analysis result for connecting pathomics data and a gene module according to an embodiment.

FIG. 4 is a diagram visually representing a connection relationship between pathomics data and a gene module according to an embodiment.

FIG. 5 and FIG. 6 are examples of enrichment analysis results for a gene module coded with a color name of black.

FIG. 7 and FIG. 8 are example diagrams showing enrichment analysis results for a gene module coded with a color name of yellow.

FIG. 9 is an example interface screen on which interpretation information is visually displayed, according to an embodiment.

FIG. 10 is a flowchart showing a method for providing interpretation information of pathomics data according to an embodiment.

FIG. 11 is a hardware configuration diagram of a computing device according to an embodiment.

### DETAILED DESCRIPTION OF THE EMBODIMENTS

Hereinafter, embodiments of the present disclosure will be described in detail with reference to the attached drawings so that the person of ordinary skill in the art may easily implement the present disclosure. The present disclosure may be modified in various ways and is not limited thereto. In the drawings, elements irrelevant to the description of the present disclosure are omitted for clarity of explanation, and like reference numerals designate like elements throughout the specification.

Throughout the specification, when a part is referred to “include” a certain element, it means that it may further include other elements rather than exclude other elements, unless specifically indicates otherwise. In addition, the term such as “ . . . unit”, “ . . . block”, “ . . . module”, or the like described in the specification mean a unit that processes at least one function or operation, which may be implemented with a hardware, a software or a combination thereof.

Until now, most researches for interpreting pathomics data (mostly, the number of cells) are performed mainly by inferring the meaning of pathomics data through correlation analysis with a single gene. Here, in order to define the correlation, a variety of arbitrary conditions are used. However, the correlation analysis between pathomics data and genes has problems as follows. First, it is difficult to set a threshold that can define related genes among about 20,000 genes. Second, it is so difficult to find biological meaning of variables that are generated according to each tissue type and/or cell type included in the histopathology data, and thus interpretation of cells in any tissue type and/or cell type is not possible. Third, it is difficult to relate the pathomics data with previously known clinical knowledge such as disease mechanisms, drug response and the like.

Hereinafter, a method of relating various histological data with genetic information, and annotating biological and/or medical interpretation information to the various histological data thereby is described. First, a description of some databases that may be used to annotate biological and/or medical interpretation information will be followed.

Biological process terms of gene ontology may be used. The biological process refers to a process genetically programmed to make an organism accomplish specific biological purpose. The biological process is a whole process generating two daughter cells from a single mother cell through, for example, cell division.

Molecular function terms of gene ontology may be used. The molecular functional terms describe functions corresponding to all processes regulating catalysis, binding, biological activity, rate, and the like that occur at the molecular level.

KEGG pathway is a database of route maps explaining knowledge of interactions among molecules, reactions, and relation network of molecules. The KEGG pathway provides representative seven biological/medical mechanisms in the form of pathway map. The KEGG pathway contains details of metabolism, genetic information processing, environmental information processing, cellular processes, organismal systems, human diseases, and drug development, and includes pathway maps of molecular networks for each subset under each category.

BIOCARTA is a database about relationships such as molecular interactions, reactions, and the like. Like the KEGG pathway, the BIOCARTA introduces specific mechanisms through molecular relationships.

The genetic association database (GAD) is a relational database of disease and genome. The GAD is a database of open genetic association studies, which contains biological/medical information about diseases, genomes, genes, and mutations for the purpose of human-genetic association studies. Therefore, the database may be modified as describing relationships between diseases and genes by shortening information in the unit of gene, and finally may perform functional enrichment analysis along with a module that is a result of the present disclosure.

Online Mendelian inheritance in man (OMIM) is a database of human genes and genetic disorders. OMIM is a database containing information about all genetic disorders, such as Mendelian disease, and may define the relationship between diseases and histologic components through correlations between diseases and modules and correlations between module and histologic components.

UniProt Keywords is a database of keywords related to proteins. UniProt Keywords has 10 sub-categories in the keywords that are constructed as a database for proteins. The 10 sub-categories are classified as biological process, cellular component, coding sequence diversity, developmental stage, disease, domain, ligand, molecular function, post-translational modification, and technical term. Each protein is a product of a gene, and many proteins may be shortened as specific genes. Namely, the UnitProt keyword can be substituted for a keyword describing a specific gene, which enables a functional enrichment analysis with the module.

UniProt tissue specificity is a database providing information on gene expression at mRNA level or at protein level in a cell or a tissue of a multicellular organism. UniProt tissue specificity is a database containing information on a specific tissue where gene is expressed. From Uniprot tissue specificity, information on tissues where each module is specifically expressed may be obtained.

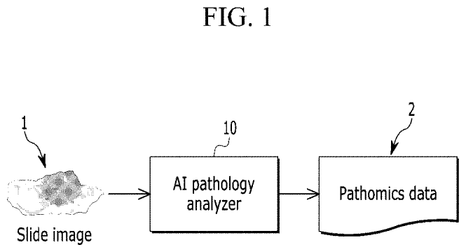

FIG. 1 is a diagram for explaining an AI pathology analyzer according to an embodiment.

Referring to FIG. 1, the AI pathology analyzer 10 is a computing device trained to receive a slide image 1 obtained through scanning diagnostic target tissue with whole slide imaging (WSI) technique, and to extract a variety of pathomics data 2 from the slide image 1. Here, the slide image 1 represents a cross section of tissue obtained from primary tumor of a patient through biopsy or surgery, and may be referred to as a pathological image. The pathomics data 2 includes information obtained through cell classification, tissue classification, and structure classification of the slide image 1 in the AI pathology analyzer 10.

The slide image 1 is produced to satisfy input conditions of the AI pathology analyzer 10. The slide image is obtained by converting a glass slide to a digital image through whole slide imaging. In order to obtain glass slides, various biopsy methods slides may be used. For example, needle biopsy, surgical biopsy, aspiration biopsy, skin biopsy, prostate biopsy, kidney biopsy, liver biopsy, bone marrow biopsy, bone biopsy, CT-guided biopsy, ultrasound-guided biopsy, and the like may be used, but the biopsy methods are not limited thereto.

The AI pathology analyzer 10 may be trained with various types of slide images, and may output AI analysis data for various cancer types and quantitative data obtained by digitizing extracted features as the number, the total amount, and the like, as the pathomics data. For example, the pthomics data may be digitized as the number of lymphoplasma cells located in cancer epithelial and cancer stroma, the total amount of cancer epithelial and cancer stroma, and the like.

Specifically, the pthomics data may include features on area information in the slide image, such as cancer epithelial, cancer stroma, normal epithelial, normal stroma, necrosis, fat, background and the like. The phthomics data may include cell classification data obtained by structurally and/or systematically classifying cells in the slide image, and digitized quantitative data. The types of cells may be variously classified, such as a degenerated tumor cell, a necrotic tumor cell, an endothelial cell, a pericyte, a mitosis, a macrophage, a lymphoplasma cell, a fibroblast, and the like. The pathomics data may include features of a specific type of cancer. For example, the features may include features indicating anomaly of breast cancer cells, such as nuclear grade 1, nuclear grade 2, nuclear grade 3, tubule formation count, tubule formation area, ductal carcinoma in situ (DCIS) count, DCIS area, and the like. Further, the pathomics data may include nerve count, nerve area, blood vessel count, blood vessel area, and the like.

The AI pathology analyzer 10 may be implemented through a machine learning model that can extract meaningful features from an image. The AI pathology analyzer 10 may include separately trained models according to a diagnosis type (e.g., cancer type). For example, the AI pathology analyzer 10 may be implemented with a deep learning-based training model such as a convolutional neural network, a graph neural network, and the like. Alternatively, the AI pathology analyzer 10 may be implemented with a relatively simple classification model such as a support vector machine (SVM), a random forest, a regression model, and the like. Needless to say, the AI pathology analyzer 10 may be implemented as a combination of various machine learning models.

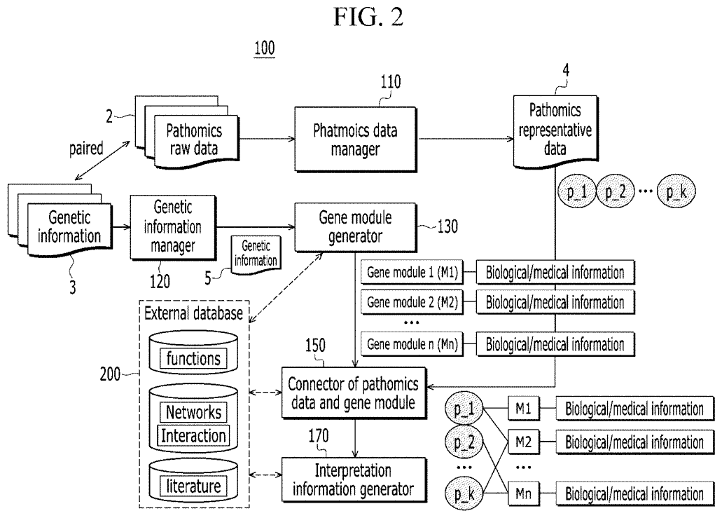

FIG. 2 is a block diagram illustrating a system for providing interpretation information of pathomics data according to an embodiment.

Referring to FIG. 2, a system for providing interpretation information of pathomics data (hereinafter, referred to as an “interpretation information providing system”) 100 may provide biological and/or medical interpretation information of pathomics data extracted from a slide image. The interpretation information providing system 100 may include the AI pathology analyzer 10 shown in FIG. 1, but, in the following description, pathomics data output from the AI pathological analyzer 10 is described as to be input to the interpretation information providing system 100. The interpretation information providing system 100 may operate independently from the AI pathology analyzer 10 and may provide interpretation information about an external AI pathology analyzer by interworking with various types of external AI pathology analyzers.

The interpretation information providing system 100 includes phtomics data manager 110, genetic information manager 120, gene module generator 130, connector between pathomics data and gene module (hereinafter, referred to as a “connector”) 150, and an interpretation information generator 170. For explanation, each component of the interpretation information providing system 100 is referred to as the pathomics data manager 110, the genetic information manager 120, the gene module generator 130, the connector 150, and the interpretation information generator 170, respectively, but may be implemented as a computing device executed by at least one processor. Here, the components may be implemented in a computing device all together or implemented as distributed in separate computing devices. When implemented in separate computing devices, each component may communicate with each other via a communication interface. A device that can execute a software program designed to perform the embodiments of the present disclosure will suffice the computing device.

The interpretation information providing system 100 interworks with various databases 200 required by the gene module generator 130, the connector 150, and the interpretation information generator 170. The various databases 200 includes a knowledge database and a literature database. The various databases may include a biological database containing genetic feature information such as relationship information between biologically discovered genes and functions, pathways, interactions, and the like, and a medical database used in medical fields such as biochemistry, medicine, pharmacy, and the like.

Biological databases providing genetic feature information may include, for example, a protein-protein interaction (PPI) network, a gene co-expression network, a gene regulatory network, a metabolic network, a system biology database, a protein-protein interaction database, a gene ontology database, a gene-gene interaction database, a synthetic biology database, a genetic interaction database, a gene set enrichment analysis (GSEA), a KEGG Pathway, BIOCARTA, UniProt Keywords, UniProt Tissue specificity, and the like.

The medical database may be a database utilized in biomedical field and may be, for example, a chemical interaction database, a disease-gene database, a gene-drug database, a gene-phenotype database, a pharmaco-genomics database, a gene-pharmacokinetic database, a gene-pharmacodynamics database, a drug-drug database, a biological pathway database, UniProt protein database, a protein domain, a protein interaction, a tissue expression, genetic association database (GAD), Online Mendelian inheritance in man (OMIM), and the like. The medical database may include a knowledge database and literature that can cluster genes and proteins.

In addition, the database may be Uniprot Sequence Feature (UP_SEQ_FEATURE), NCBI's COG database (COG_ONTOLOGY), PUBMED Literature ID, REACTOME pathways, biological biochemical image database (BBID), EMBL-EBI InterPro, EMBL-EBI IntAct, simple modular architecture research tool (SMART), protein information resource (PIR), BIOGRID database, and the like.

The interpretation information providing system 100 receives analysis data where pathomics data 2 of a patient is paired with genetic information 3. The pathomics data 2 is raw data that is input to the phatomics data manager 110. The genetic information 3 is raw data that is input to the genetic information manager 120.

The pathomics data 2 is data output from the AI pathology analyzer 10 that receives the slide image 1 of the patient, as shown in FIG. 1. As such, the interpretation information providing system 100 receives samples of a plurality of patients, and the pathomics data samples and the genetic information samples are paired. It is assumed that the interpretation information providing system 100 receives pathomics data and genetic information of a patients cohort. The patients cohort refers to a group of patients diagnosed with a specific disease, and pathomics data and genetic information of patients of the same disease are used.

Genetic information 3 is biological information quantified such as transcriptome, proteome, and the like. For example, the genetic information 3 may include RNA information and/or protein information, which are product of gene expression. In the present disclosure, the terms RNA and protein may be used without distinction. Gene information 3 may include quantitative data of RNA and/or protein. The genetic information manager 120 may generate or modify genetic information according to the input condition of the gene module generator 130. Genetic information 3 may be generated as a gene/protein set having a specific function by the gene module generator 130.

Quantitative data of RNA may be numerically measured data of the amount of genes expressed to mRNA state. RNA quantitative data may be obtained by a transcriptomics technique that measures gene-expressed RNA. As a transcriptomics technique, for example, apolymerase chain reaction (PCR), real-time PCR (qPCR), microarray, NGS RNA sequencing, targeted RNA seqeuencing, and the like may be used.

Protein quantitative data is numerically measured data of expression of a protein having a function. The protein quantitative data may be obtained by a proteomics technique. As a proteomics technique, for example, reverse phase protein array (RPPA), mass spectrometry, blotting techniques for protein quantification, and the like may be used.

The pathomics data 2 includes data numerically quantified information of a tissue and a cell contained in the slide image. That is, the pathomics data 2 is a quantified value as the number of cells or pixels that are counted in cells, tissues, and structures.

The pathomics data output from a Lunit SCOPE may be coded, for example, as shown in Table 1. In table 1, CE and CS may refer to cancer epithelial and cancer stroma, respectively. Each code may be abbreviation of the names of the tissue/cell. For example, CE stands for cancer epithelium, CS stands for cancer stroma, NE stands for normal epithelium, NS stands for normal stroma, N stands for necrosis, F stands for fat, PC stands for endothelial cell and pericyte, MTS stands for mitosis, MA stands for macrophage, TIL stands for lymphoplasma cell, FB stands for fibroblast, N1 stands for Nuclear grade 1, N2 stands for Nuclear grade 2, N3 stands for Nuclear grade 3, TB stands for tubule formation, DCIS stands for ductal carcinoma in situ (DCIS), NV stands for nerve, and BV stands for blood vessel. PER and DEN stands for percentage and density, respectively. Each code can be used for interpret the meaning of the data.

TABLE 1

| No. | Pathomics | Description |
|---|---|---|
| P1 | CE_PER | Percentage of the number of cellscorrespondingto cancer |
| | | epithelium to that of cells existing in the entire image area |
| P2 | CS_PER | Percentage of thenumber of cells correspondingto cancer stroma |
| | | to that of cells existing in the entire image area |
| P3 | NE_PER | Percentageof the number of cellscorrespondingto normal |
| | | epithelium to that of cells existing in the entire image area |
| P4 | NS_PER | Percentage of the number of cells corresponding to normal stroma |
| | | to that of cells existing in the entire image area |
| P5 | CE_PC_PER | Percentage of endothelial cells and pericyte type cells to cells |
| | | existing in an area of cancer epithelium |
| P6 | CE_PC_DEN | Density of endothelial cells and pericyte type cells among cells |
| | | existing an area of cancer epithelium |
| P7 | CS_PC_PER | Percentage of endothelial cells and pericyte type cells among cells |
| | | existing in an area of cancer stroma |
| P8 | CS_PC_DEN | Density of endothelial cells and pericyte type cells among cells |
| | | existing in an area of cancer stroma |
| P9 | NE_PC_PER | Percentage of endothelial cells and pericyte type cells to cells |
| | | existing in an area of normal epithelium |
| P10 | NE_PC_DEN | Density of endothelial cells and pericyte type cells among cells |
| | | existing in an area of normal epithelium |
| P11 | NS_PC_PER | Percentage of endothelial cells and pericyte type cells among cells |
| | | existing in an area of normal stroma |
| P12 | NS_PC_DEN | Density of endothelial cells and pericyte type cells among cells |
| | | existing in an area of normal stroma |
| P13 | CE_MTS_PER | Percentage of cells in mitosis state among cells existing in an area |
| | | of cancer epithelium |
| P14 | CE_MTS_DEN | Density of cells in mitosis state existing in an area of cancer |
| | | epithelium |
| P15 | CS_MTS_PER | Percentage of cells in mitosis state among cells existing in an area |
| | | of cancer stroma |
| P16 | CS_MTS_DEN | Density of cells in mitosis status existing in an area of cancer stroma |
| P17 | NE_MTS_PER | Percentage of cells in mitosis state among cells existing in an area |
| | | of normal epithelium |
| P18 | NE_MTS_DEN | Density of cells in mitosis state existing in an area of normal |
| | | epithelium |
| P19 | NS_MTS_PER | Percentage of cells in mitosis state existing in an area of normal |
| | | stroma |
| P20 | NS_MTS_DEN | Density of cells in mitosis state existing in an area of normal stroma |
| P21 | CE_MA_PER | Percentage of macrophage type cells against cells existing in an |
| | | area of cancer epithelium |
| P22 | CE_MA_DEN | Density of macrophage type cells existing in an area of cancer |
| | | epithelium |
| P23 | CS_MA_PER | Percentage of macrophage type cells existing in an area of cancer |
| | | stroma |
| P24 | CS_MA_DEN | Density of macrophage type cells existing in an area of cancer |
| | | stroma |
| P25 | NE_MA_PER | Percentage of macrophage type cells existing in an area of normal |
| | | epithelium |
| P26 | NE_MA_DEN | Density of macrophage type cells existing in an area of normal |
| | | epithelium |
| P27 | NS_MA_PER | Percentage of macrophage type cells existing in an area of normal |
| | | stroma |
| P28 | NS_MA_DEN | Density of macrophage type cells existing in an area of normal |
| | | stroma |
| P29 | CE_TIL_PER | Percentage of lymphoplasma cell type cells existing in an area of |
| | | cancer epithelium |
| P30 | CE_TIL_DEN | Density of lymphoplasma cell type cells existing in an area of |
| | | cancer epithelium |
| P31 | CS_TIL_PER | Percentage of lymphoplasma cell Type cells existing in an area of |
| | | cancer stroma |
| P32 | CS_TIL_DEN | Density of lymphoplasma cell type cells existing in an area of |
| | | cancer stroma |
| P33 | NE_TIL_PER | Percentage of lymphoplasma cell type cells existing in an area of |
| | | normal epithelium |
| P34 | NE_TIL_DEN | Density of lymphoplasma cell type cells existing in an area of |
| | | normal epithelium |
| P35 | NS_TIL_PER | Percentage of lymphoplasma cell type cells existing in an area of |
| | | normal stroma |
| P36 | NS_TIL_DEN | Density of lymphoplasma cell type cells existing in an area of |
| | | normal stroma |
| P37 | CE_FB_PER | Percentage of fibroblast type cells existing in an area of cancer |
| | | epithelium |
| P38 | CE_FB_DEN | Density of fibroblast type cells existing in a region of cancer |
| | | epithelium |
| P39 | CS_FB_PER | Percentage of fibroblast type cells existing in an area of cancer |
| | | stroma |
| P40 | CS_FB_DEN | Density of fibroblast type cells existing in an area of cancer stroma |
| P41 | NE_FB_PER | Percentage of fibroblast type cells existing in an area of normal |
| | | epithelium |
| P42 | NE_FB_DEN | Density of fibroblast type cells existing in an area of normal |
| | | epithelium |
| P43 | NS_FB_PER | Percentage of fibroblast type cells existing in an area of normal |
| | | stroma |
| P44 | NS_FB_DEN | Density of fibroblast type cells existing in an area of normal stroma |
| P45 | CE_N1_PER | Percentage of cells in nuclear grade 1 state existing in an area of |
| | | cancer epithelium |
| P46 | CE_N1_DEN | Density of cells in nuclear grade 1 state existing in an area of |
| | | cancer epithelium |
| P47 | CE_N2_PER | Percentage of cells in nuclear grade 2 state existing in an area of |
| | | cancer epithelium |
| P48 | CE_N2_DEN | Density of cells in nuclear grade 2 state existing in an area of |
| | | cancer epithelium |
| P49 | CE_N3_PER | Percentage of cells in nuclear grade 3 state existing in an area of |
| | | cancer epithelium |
| P50 | CE_N3_DEN | Density of cells in nuclear grade 3 state existing in an area of |
| | | cancer epithelium |
| P51 | CE_TB_DEN_CNT | Density of the number of tubule formation tissue type cells existing |
| | | in an area of cancer epithelium |
| P52 | CE_TB_DEN_AREA | Density of area of tubule formation tissue type cells existing in an |
| | | area of cancer epithelium |
| P53 | CE_DCIS_DEN_CNT | Density of the number of ductal carcinoma in situ (DCIS) tissue |
| | | type cells existing in an area of cancer epithelium |
| P54 | CE_DCIS_DEN_AREA | Density of a region of ductal carcinoma in situ (DCIS) tissue type |
| | | cells existing in an area of cancer epithelium |
| P55 | CE_BV_DEN_CNT | Density of the number of cells corresponding to blood vessel |
| | | existing in an area of cancer epithelium |
| P56 | CE_BV_DEN_AREA | Density of the cell area corresponding to blood vessel existing in |
| | | an area of cancer epithelium |
| P57 | CS_BV_DEN_CNT | Density of the number of cells corresponding to blood vessel |
| | | existing in an area of cancer stroma |
| P58 | CS_BV_DEN_AREA | Density of the cell area corresponding to blood vessel existing in |
| | | an area of cancer stroma |
| P59 | NE_BV_DEN_CNT | Density of the number of cells corresponding to blood vessel |
| | | existing in an area of normal epithelium area |
| P60 | NE_BV_DEN_AREA | Density of cell area corresponding to blood vessel existing in an |
| | | area of normal epithelium |
| P61 | NS_BV_DEN_CNT | Density of the number of cells corresponding to blood vessel |
| | | existing in an area of normal stroma |
| P62 | NS_BV_DEN_AREA | Density of cell area corresponding to blood vessel existing in an |
| | | area of normal stroma |
| P63 | N1_PER | Percentage of the number of cells in nuclear grade 1 state to that |
| | | of cells existing in the entire image area |
| P64 | N2_PER | Percentage of the number of cells in nuclear grade 2 state to that |
| | | of cells existing in the entire image area |
| P65 | N3_PER | Percentage of the number of cells in nuclear grade 3 state to that |
| | | of cells existing in the entire image area |

Hereinafter, a description of the pathomics data manager 110 will be followed.

The pathomics data manager 110 preprocesses input pathomics raw data 2 and stores the preprocessed pathomics data.

The pathomics data manager 110 may classify parameters constituting the pathomics data into tissue information and cell information, and may remove quantitative data of information on a cell type that cannot exist in a tissue or on features that are not discovered, from each pathomics data, based on a relationship table between tissue information and cell information.

For example, the relationship table between tissue information and cell information is composed of a relationship matrix between tissue and cells as shown in Table 2, and information of cells to be removed from each tissue is mapped thereto. In Table 2, the tissue information is written on the horizontal axis. Here, CE stands for cancer epithelium, CS stands for cancer stroma, NE stands for normal epithelium, NS stands for normal stroma, N stands for necrosis, and F stands for Fat. In Table 2, the cell information is written in the vertical axis. Here, PC stands for Endothelial cell and pericyte, MTS stands for mitosis, MA stands for macrophage, TIL stands for lymphoplasma cell, FB stands for fibroblast, N1 stands for nuclear grade 1, N2 stands for nuclear grade 2, N3 stands for nuclear grade 3, TB stands for tubule formation, DCIS stands for ductal carcinoma in situ (DCIS), NV stands for nerve, and BV stands for blood vessel.

TABLE 2

| Tissue cell | CE | CS | NE | NS | N | F |
|---|---|---|---|---|---|---|
| PC | | | | | x | x |
| MTS | | | | | x | x |
| MA | | | | | x | x |
| TIL | | | | | x | x |
| FB | | | | | x | x |
| N1 | | x | x | x | x | x |
| N2 | | x | x | x | x | x |
| N3 | | x | x | x | x | x |
| TB | | x | x | x | x | x |
| DCIS | | x | x | x | x | x |
| NV | x | x | x | x | x | x |
| BV | | | | | x | x |

Cancer cells are very rare in an adipose tissue. Accordingly, the number of cells annotated with information about nuclear grade may be wrong or not helpful for predicting the features of carcinoma at all. Therefore, if cell feature values (that is, PC, MTS, BV, etc.) are counted on the adipose tissue F in the pathomics raw data, the pathomics data manager 110 removes the corresponding values referring to Table 2. If feature values of target cell to be removed are counted on tissues (CE, CS, NE, NS, N) classified from each pathomics raw data, the pathomics data manager 110 removes the corresponding values as the case of the adipose tissue F.

Additionally, the pathomics data manager 110 may remove a parameter having a small count value from the pathomics raw data. In pathomics data that is quantitative data, since a very small value affects statistical analysis due to a fold having a large variation, the pathomics data manager 110 filters out cell feature values with meaningless distributions or small values. The pathomics data manager 110 may find a cell feature corresponding to an outlier in the entire sample, for example, in the way of count per million (CPM).

The pathomics data manager 110 calculates representative values of individual data constituting the pathomics data, by using pathomics data obtained through preprocessing each pathomics raw data 2. The individual pathomics data may be the number of specific cells or tissues, or the number of pixels of specific cells or tissues. The specific cells or tissues may be, for example, endothelial cell and pericyte, and mitosis (MTS). Further, the individual pathomics data simply may be a single parameter constituting the pathomics data and may be referred to as a “p (pathomics) feature” or a “p feature cell” in the description.

It is assumed that a plurality of samples (e.g., K samples) is input to pathomics data manager 110. Then, the pathomics data manager 110 calculates a representative value representing K samples for each p feature.

The way the pathomics data manager 110 calculates a representative value for each p feature may be various. For example, the pathomics data manager 110 may use a relative log cell-count (RLC)-based data normalization method. An expected p feature value E[Yₚₖ] of k samples among K samples may be defined by Equation 1.

$$E [ {Y}_{pk} ] = \frac{{μ}_{pk}}{{s}_{k}} {N}_{k} \\ {S}_{k} = {\sum }_{p = 1}^{P} {μ}_{pk} \qquad ( Equation 1 )$$

In Equation 1, Yₚₖ is a count level of p feature cells measured in k samples (pathological image), and E[Yₚₖ] is an distribution of p feature cells expected from Yₚₖ. Nₖ is a count level of all cells or pixels measured in k samples. μₚₖ is a correct answer and an actual count level of p feature cells for unknowable K samples. Sₖ is an actual count level of all cells for k samples.

A pseudo-reference Yₚ^{RLC} representing K samples may be defined by Equation 2. In Equation 2, r is a biological replicate. In Equation 2, Xₚᵣₖ is a count of p feature and r for k samples.

$${Y}_{p}^{RLC} = \sqrt[kr]{{Π}_{k = 1}^{K} {Π}_{r = 1}^{R} {X}_{prk}} \qquad ( Equation 2 )$$

The pathomics data manager 110 may normalize p feature value, through dividing the p feature value Xₚᵣₖ by a scaling factor Yₚ^{RLC}. The scaling factor makes a distribution of quantitative data be normalized.

The pathomics data manager 110 may remove left skewed characteristic from the count data by posing Log₂( ) on the normalized p feature representative value.

Through the above-described processes, the pathomics data manager 110 generates pathomics representative data 4 which represents the pathomics data including K samples. The pathomics representative data 4 may be expressed as a set of p features, and each p feature has a representative value which is a quantitative data.

Next, a description about the genetic information manager 120 will be followed.

The genetic information manager 120 may remove down-regulated genes from all gene samples. The genetic information manager 120 may find cell feature corresponding to an outlier sample in all samples, by a count per million (CPM) method. If a gene having a CPM value less than 1 is more than or equal to half of all samples, the gene may be defined as a down-regulated gene and may be excluded. In other words, in the genetic information (e.g., RNA sequence) that is quantitative data, since a very small value affects statistical analysis, the corresponding value is analytically filtered out. The CPM (C_{gk}) of g gene of the k-th sample may be defined by Equation 3.

C_{gk}=(μ_{gk}/Y_{gk})*1000000 (Equation 3)

In Equation 3, Y_{gk} is a read count of g gene in k samples, and μ_{gk} is an expression level of the g gene in k samples.

The genetic information manager 120 extracts genetic information from a plurality of samples (e.g., K samples). Here, an arbitrary specific gene may be referred to as “g gene”. The genetic information manager 120 may utilize various techniques to calculate information of the g gene.

The genetic information manager 120 may use various data normalization methods to obtain the genetic information of the g gene. For example, at least one of a data normalization technique based on relative log-expression (RLE) and a data normalization technique based on trimmed mean of M value may be used.

According to an embodiment, the genetic information manager 120 may use a data normalization technique based on relative log-expression (RLE). An expected g expression value E[Y_{gk}] in k samples of the K samples may be defined by Equation 4. Since Y_{gk} is the number of read counts of the g gene measured in k samples and is merely a partial sequence read count, it is possible to predict the actual expression value E[Y_{gk}] from Y_{gk}.

$$E [ {Y}_{gk} ] = \frac{{μ}_{gk} {L}_{g}}{{s}_{k}} {N}_{k} \\ {S}_{k} = {\sum }_{g = 1}^{G} {μ}_{gk} {L}_{g} \qquad ( Equation 4 )$$

In Equation 4, L_{g} is a length of the g gene, and Nₖ is the number of read counts of the entire gene measured in k samples.

A pseudo-reference Y_{g}^{RLE} representing K samples may be defined by Equation 5. In Equation 5, r is biological replicate, and X_{grk} is a read count for the g gene and r in k samples.

$${Y}_{g}^{RLE} = \sqrt[kr]{{\prod }_{k = 1}^{K} {{\prod }_{r = 1}}^{R} {x}_{grk}} \qquad ( Equation 5 )$$

The genetic information manager 120 may normalize a distribution of g expression value by dividing the g expression value X_{grk} with a scaling factor Y_{g}^{RLE}. The scaling factor has an effect of normalizing a distribution of quantitative data.

According to another embodiment of the present disclosure, the genetic information manager 120 may use a normalization technique based on trimmed mean of M value. Among the genetic information, RNA-sequencing data is composed of reads. The sizes of gene samples are different, and each gene has different library composition. Thus, the genetic information manager 120 may normalize the size of the gene samples.

First, the genetic information manager 120 selects a reference sample K ‘ among K samples. Then, the genetic information manager 120 obtains an M-value M_{g} corresponding to log-fold for the reference sample K’, for all of K samples. For example, M_{g} may be defined by Equation 6.

$${M}_{g} = {log}_{2} \frac{{Y}_{gk} / {N}_{k}}{{Y}_{{gk}^{′}} / {N}_{{k}^{′}}} \qquad ( Equation 6 )$$

The genetic information manager 120 obtains an A-value A_{g} corresponding to a geometric mean of the reference sample K′ and the k-th sample. The A value A_{g}, for example, may be defined by Equation 7. The A value A_{g} may be defined by an absolute expression level.

A_{g}=½log₂(Y_{gk}/Nₖ*Y_{gk′}/N_{k′}) (Equation 7)

M-value M_{g} being a log fold change is a reference value for finding a biased gene, and A-value A_{g} being a geometric mean is a reference value for finding up-regulated/down-regulated genes. The genetic information manager 120 may remove genes that fall within the upper/lower 30% of the M-value and genes having upper 5% of A-value, and determine a scaling value normalizing the size of the gene samples through the remaining genes. That is, the genetic information manager 120 may determine a scaling factor by using a trimmed mean, and normalize the size of each gene sample by dividing the library size of each gene sample with the scaling factor.

So far, two data normalization techniques based on relative log-expression (RLE) and based on trimmed mean of M value have been described as examples of data normalization techniques used by the genetic information manager 120. To select which of the two techniques depends on the number of independent variables. The data normalization technique based on RLE may be used for data having a small number of independent variables, and a data normalization technique based on trimmed mean of M value may be used for data affected by outlier values due to having a large number of independent variables.

Through such a procedure, the genetic information manager 120 generates genetic information 5 from the genetic information of the K samples. Genetic information may be expressed as a set of g genes.

Hereinafter, a description of the gene module generator 130 will be followed.

The gene module generator 130 receives the gene information 5 generated by the genetic information manager 120. The gene module generator 130 generates at least one gene module related to the genetic information 5 by using quantitative data of RNAs and/or proteins included in the genetic information 5. A gene module is a group containing correlated genes or a group containing genes having similar functions. Further, the gene module may be composed of a single RNA/single protein. The gene module generator 130 may give a biological and/or medical meaning to the gene module through biological and/or medical information annotated to multiple genes included in each gene module.

The gene modules may be generated in various ways. According to an embodiment, based on a statistical technique, the gene module generator 130 searches for a correlation network of data included in the genetic information 5 using De-novo, whereby correlated genes may be modularized into a same group. According to another embodiment, the gene module generator 130 may extract correlated genes based on unsupervised machine learning and may modularize the extracted genes into a same group. According to still another embodiment, the gene module generator 130 may use gene function groups defined in an external database. That is, a plurality of gene modules exists in the form of a predefined functional group, and the gene module generator 130 may extracts at least one gene module including genes contained in the gene information 5 from the plurality of gene modules.

Hereinafter, an example of an extraction method of a gene module through a correlation network will be described.

First, the gene module generator 130 generates a correlation network connecting genes based on interactions of the genes included in the genetic information 5. A node in the correlation network is a gene, and an edge represents an interaction between connected genes. Interactions among all genes may be determined by pairwise-correlation between two genes. For example, gene interactions (dependencies) may be confirmed through rank correlations such as Pearson's correlation coefficient, Sperman's rank coefficient, Kendall tau rank correlation, and the like. An equation aᵢⱼ=|cor(xᵢxⱼ)|^{β} (here, i and j are indices of genes) represents a correlation between genes when using a correlation threshold of β, and the interactions among n genes may be calculated with an n×n matrix, if the total number of genes is n.

Gene module generator 130 makes clusters of genes having the same functions in the correlation network. Since a gene or a protein with a large topological overlap value is known to have a high probability of having the same functions, the gene module generator 130 may extract genes having the same function by calculating the topological overlap value in the correlation network. The topological overlap value corresponds to interconnectedness between two genes. The topological overlap value tᵢⱼ of the i-gene and j-gene may be calculated by Equation 8.

$${t}_{ij} = \frac{\vert {N}_{1} ( i ) ⋂ {N}_{1} ( j ) \vert + {a}_{ij}}{min \{ \vert {N}_{1} ( i ) \vert , \vert {N}_{1} ( j ) \vert \} + 1 - {a}_{ij}} \qquad ( Equation 8 )$$

In Equation 8, when i and j are equal (that is, i=j), “a” is 1. N₁(i) refers to genes directly connected to the i gene (gene nodes having a distance of 1 from i gene node), and |⋅| means the number of included genes.

The gene module generator 130 generates a gene module by clustering genes with a high probability of having the same function, by using a topological overlap value. Here, the gene module generator 130 calculates a distance Dᵢⱼ between two genes based on the interconnection value tᵢⱼ between the two genes obtained by the topological overlap, and performs hierarchical clustering for the genes based on the distance. Through clustering, a plurality of gene modules may be generated. Various techniques such as k-means clustering, consensus clustering, and the like, may be used for clustering.

The gene module generator 130 extracts representative information of the plurality of gene modules. The gene module generator 130 may extract representative information representing genes existing in each gene module, by using principal component analysis (PCA). The representative information of each gene module may be a first PCA vector, which may be defined as an eigengene of each gene module.

When a plurality of gene modules related to the gene information 5 is determined, the gene module generator 130 determines biological functions significantly enriched in each gene module through functional enrichment analysis. Additionally, when a plurality of gene modules related to the gene information 5 is determined, the gene module generator 130 may add biological information and medical information describing each gene module with reference to accessible databases and literature.

First, the gene module generator 130 may extract a specific function in which the representative information of each gene module is significantly enriched, among functions defined in an external database. Here, the gene module generator 130 may use gene set enrichment analysis (GSEA). For example, from external databases of gene ontology (GO) and Kyoto encyclopedia of genes and genomes (KEGG), the gene module generator 130 may extract functions of gene ontology (e.g., immune response, immune system process, etc.) and KEG functions (e.g., cytokine-cytokine receptor interaction, etc.), where any gene module is significantly enriched.

The gene module generator 130 may perform significance test on association of the extracted specific function corresponding to each gene module. Here, various significance test method such as Fisher's exact test, chi square test, cochran test, and the like may be used. If the functions extracted corresponding to each gene module are plural, the gene module generator 130 may annotate a plurality of functions to the corresponding gene module, and set a representative function that is displayed preferentially.

For example, the plurality of gene modules may be coded with color names, and mapped to functional information, as shown in Table 3.

TABLE 3

| | | Classified genetic | |
|---|---|---|---|
| | | information | |
| No. | Gene module | (Example) | Function |
| M1 | Black | SPNS2, FAM153A, | immune response, immune system |
| | | RRN3P1, ZNF57, | process, regulation of immune system |
| | | BHLHE22, NCF1C, | process, defense response, leukocyte |
| | | SCML4, LILRB1, GM2A, | activation |
| | | SYAP1 | |
| M2 | Yellow | MYLK2, FBX043, | mitotic cell cycle, mitotic cell cycle |
| | | GDPD2, GOLT1B, | process, cell cycle, cell cycle process, |
| | | WHAMML2, NHLH2, | chromosome organization |
| | | CABLES2, PBK, CEP152, | |
| | | LAMB2 | |
| M3 | Yellowgreen | IF144, HSH2D, IL22RA1, | response to virus, defense response to |
| | | STAT2, RTP4, OASL, | virus, innate immune response, type I |
| | | TRAFD1, IFIT1, ISG15, | interferon signaling pathway, cellular |
| | | DHX58 | response to type I interferon |
| M4 | Magenta | COL11A2, HIF3A, | tissue development, single-multicellular |
| | | KRT81, ITGB8, C4BPA, | organism process, anatomical structure |
| | | EPHB1, XDH, SYNM, | development, epidermis development, |
| | | KLK8, IFF02 | multicellular organismal process |
| M5 | Lightgreen | GPR176, LPHN2, | homophilic cell adhesion via plasma |
| | | PCDH18, CDKL1, STL, | membrane adhesion molecules, cell-cell |
| | | ENTPD1, FILIP1, ITGAV, | adhesion via plasma-membrane adhesion |
| | | UTRN, KLF12 | molecules, movement of cell or |
| | | | subcellular component, vasculature |
| | | | development, blood vessel development |
| M6 | Pink | MTMR11, CHST6, | extracellular matrix organization, |
| | | FILIP1L, F13A1, ABCG4, | extracellular structure organization, |
| | | FNDC4, ISM1, LPAR1, | multicellular organism development, |
| | | ANAPC5, CCBE1 | single-multicellular organism process, |
| | | | system development |
| M7 | Cyan | SEMA3G, HTR2B, | single-multicellular organism process, |
| | | ABCB1, PRELP, | vasculature development, circulatory |
| | | ARHGAP6, CAPN11, | system development, cardiovascular |
| | | ZCCHC24, DNASE1L3, | system development, blood vessel |
| | | HOXA7, GNAL | development |
| M8 | Violet | KY, SPOCK3, PIK3C2G, | anterograde trans-synaptic signaling, |
| | | TNS4, CLDN19, TRPM3, | synaptic signaling, trans-synaptic |
| | | KLHL29, ALX4, | signaling, chemical synaptic trans- |
| | | TP53AIP1, TEPP | mission, nervous system development |
| M9 | darkslateblue | HIST2H2BA, HIST1H3G, | Systemic lupus erythematosus, |
| | | HIST1H2BG, HIST1H1E, | nucleosome organization, nucleosome |
| | | HIST1H4H, HIST1H1D, | assembly, chromatin assembly or |
| | | HIST1H2BE, | disassembly, Alcoholism |
| | | HIST1H2BH, | |
| | | HIST1H2BD, HIST1H1C | |
| M10 | Orange | TMEM196, RPS4Y1, | regulation of wound healing, regulation |
| | | GCG, MOGAT3, | of response to wounding, inorganic |
| | | UGT2A3, REG1B, | anion transport, negative |
| | | AP0A2, CDH9, | regulation of wound healing, |
| | | NCRNA00230B, 5T85IA3 | triglyceride metabolic process |
| M11 | Blue | PBXIP1, RNF13, PRKCZ, | cellular metabolic process, metabolic |
| | | DDAH2, ZNF273, UBTF, | process, cellular macromolecule |
| | | CC2D1A, BBC3, SFTPD, | metabolic process, primary metabolic |
| | | USF2 | process, organic substance metabolic |
| | | | process |
| M12 | Darkturquoise | NEU1, PPP1R11, YIF1B, | cellular nitrogen compound metabolic |
| | | CCDC86, MRPS18A, | process, mitochondrial translation, |
| | | UQCRFS1, RTN4IP1, | mitochondrial translational elongation, |
| | | MRP522, GNL1, WDR77 | mitochondrial translational termination, |
| | | | gene expression |
| M13 | royalblue | RPL36, EEF2, RPL15, | SRP-dependent cotranslational protein |
| | | HNRNPA1, EIF3M, | targeting to membrane, cotranslational |
| | | RPS14, RPS27, RPL14, | protein targeting to membrane, protein |
| | | RPS11, RPL10 | targeting to ER, establishment of protein |
| | | | localization to endoplasmic reticulum, |
| | | | nuclear-transcribed mRNA catabolic |
| | | | process, nonsense-mediated decay |
| M14 | Brown | ATL2, PVRL1, ILDR1, | ion transport, transmembrane transport, |
| | | NCRNA00094, ARL14, | ion transmembrane transport, cell |
| | | NUAK2, FAM47E, | projection organization, cell projection |
| | | TMEM144, LRGUK, | morphogenesis |
| | | KATNA1 | |
| M15 | Darkgrey | FAM171A2, TMED8, | protein localization, cellular localization, |
| | | ZNF20, MAGED1, VEZT, | establishment of localization in cell, |
| | | DTNB, ARHGEF3, | protein transport, organic substance |
| | | CYP2D6, FBX017, | transport |
| | | SNX14 | |
| M16 | bisque4 | DUSP1, TRIB1, EGR4, | positive regulation of cellular process, |
| | | GADD45B, KLF4, | cellular response to chemical stimulus, |
| | | CYR61, HBEGF, HAS1, | negative regulation of cellular metabolic |
| | | PPP1R15A, NR4A1 | process, regulation of cellular |
| | | | macromolecule biosynthetic process, |
| | | | positive regulation of cellular metabolic |
| | | | process |

Hereinafter, a description of the connector 150 will be followed.

The connector 150 extracts relationships between the representative pathomics data and the plurality of gene modules, by using various techniques. Here, the representative pathomics data is composed of a plurality of individual pathomics data, and a value of each individual pathomics data has a representative value of a plurality of samples.

The connector 150 may calculate a correlation between the representative information of the gene modules and the representative pathomics data. In this case, the representative information of the gene modules is information shortened in a designated manner, and may be shortened by various statistical methods such as an average value analysis of genes included in each gene module, a PCA, a centroid, an eigengene, and the like. The connector 150 may calculate correlations through correlation techniques such as Pearson, Spearman, kendall, and the like.

The connector 150 may determine existence of relationship between individual pathomics data and each gene module, by comparing a one-to-one relationship value between the individual pathomics data and each gene module with a threshold value (e.g., p-value). In addition to the relationship value calculated with the correlation, the connector 150 may determine the existence of the relationship between individual pathomics data and each gene module through an unsupervised clustering technique. The unsupervised clustering technique may be, for example, hierarchical clustering, consensus clustering, non-negative matrix factorization, and the like.

For example, the connector 150 may determine that each of the individual pathomics data CE_TIL_DEN and CS_TIL_DEN has a positive relationship (for example, a relationship value of 0.42 and 0.35, respectively) with a gene module corresponding to immune response and immune system process (for example, coded with a color name of black). Then, the connector 150 connects each of the individual pathomics data CE_TIL_DEN and CS_TIL_DEN with the gene module corresponding to immune response and immune system process. Further, the individual pathomics data may be connected to a plurality of gene modules.

Next, a description of the interpretation information generator 170 will be followed.

The interpretation information generator 170 receives a connection relationship between individual pathomics data and each gene module from the connector 150. The interpretation information generator 170 refers to biological function information and medical description information that are extracted corresponding to the gene module by the gene module generator 130. Further, the interpretation information generator 170 maps biological function information and medical description information extracted corresponding to the gene module as interpretation information of the individual pathomics data. The interpretation information generator 170 may provide a means to interpret the meaning of the pathomics data extracted from the phtological slide as annotated information to the gene/protein, through the biological and/or medical information of the gene module associated/correlated with the pathomics data.

The interpretation information generator 170 may provide an interface screen that visualizes digital pathology data, a gene module, and biologically and/or medically related interpretation information.

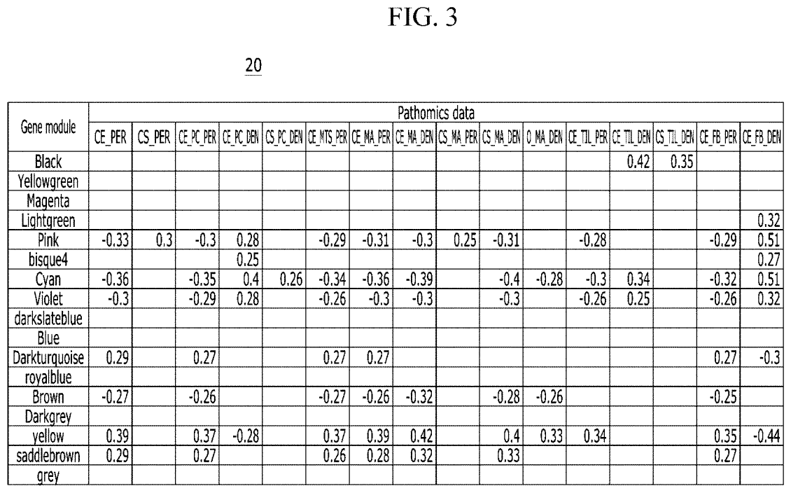

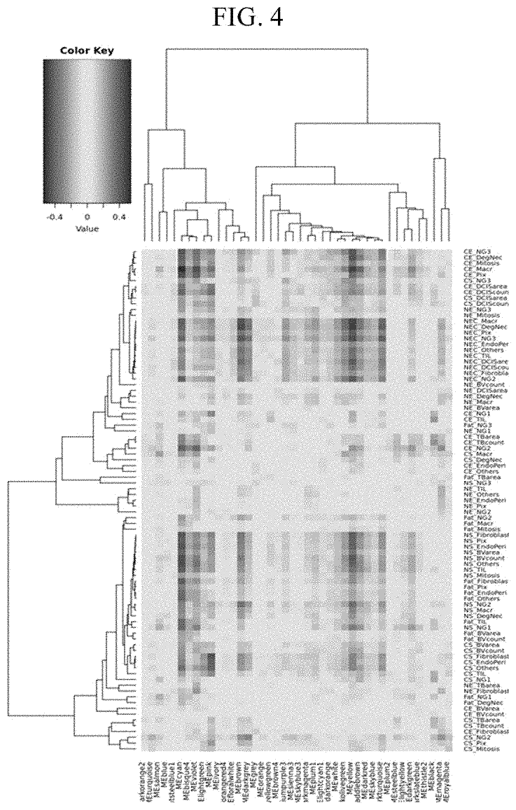

FIG. 3 is an example of a relationship analysis result for connecting pathomics data and a gene module according to an embodiment, and FIG. 4 is a diagram visually representing a connection relationship between pathomics data and a gene module according to an embodiment.

Referring to FIG. 3, the connector 150 calculates a one-to-one relationship value between a value of each gene module and individual phatomics data. The relationship value may indicate a positive or negative relationship. The connector 150 may display the relationship analysis result 20 on an interface screen. The relationship analysis result 20 is a result of correlation analysis between the pathomics data and representative information (e.g., eigenvector) of gene modules which is composed of transcript genes. In the relationship analysis result 20, each column represents a component of the pathomics data and each row represents a gene module obtained from TCGA transcript data named with an arbitrary color. In the relationship analysis result 20, each cell may be displayed only for a pair of pathomics data-gene module that is determined to have a significant correlation through Pearson correlation analysis. The correlation may be analyzed for data with both a positive correlation and a negative correlation.

Referring to the relationship analysis result 20, it is determined that CE_TIL_DEN and CS_TIL_DEN of the digital pathology data have positive relationships (e.g., relationship values of 0.42 and 0.35, respectively) with a module encoded with a color name of black.

Referring to the relationship analysis result 20, it is determined that CE_FB_DEN of the digital pathology data has positive relationships with modules coded with color names of lightgreen, pink, bisque4, and cyan, and has a negative relationship with a module encoded with a color name of yellow.

Each gene module coded with a color name is annotated with functional information significantly enriched in the gene module, and medical information describing each gene module.

For example, a gene module coded with the color name of black may be annotated with a function of immune response and immune system process of gene ontology.

A gene module coded with the color name of lightgreen may be annotated with a vessel development function of gene ontology. A gene module coded with the color name of pink may be annotated with angiogenesis and blood vessel development of gene ontology, which is a function related to vessel generation.

A gene module coded with the color name of bisque4 may be annotated with a function of cellular process metabolic process of gene ontology. A gene module coded with the color name of cyan may be annotated with an extracellular matrix organization function of gene ontology.

A gene module coded with a color name of saddlebrown is annotated with a function of protein folding and metabolic process of gene ontology

A gene module coded with the color name of yellow can be annotated with functions of cell cycle, nuclear division and DNA replication, which are functions related to cell generation of gene ontology.

Referring to FIG. 4, a connection relationship between pathomics data (shown in vertical axis, that is, Y axis) and gene modules (shown in horizontal axis, that is, X axis) may be visually displayed. Correlation values range from −0.542 to 0.491. The pathomics data may be histologic component.

In FIG. 4, a plurality of individual pathomics data that are adjacently located in the direction of Y axis may be interpreted to have similar meaning and high correlation thereamong. In addition, each gene module adjacently located in the direction of X axis may be interpreted to have similar gene expression pattern.

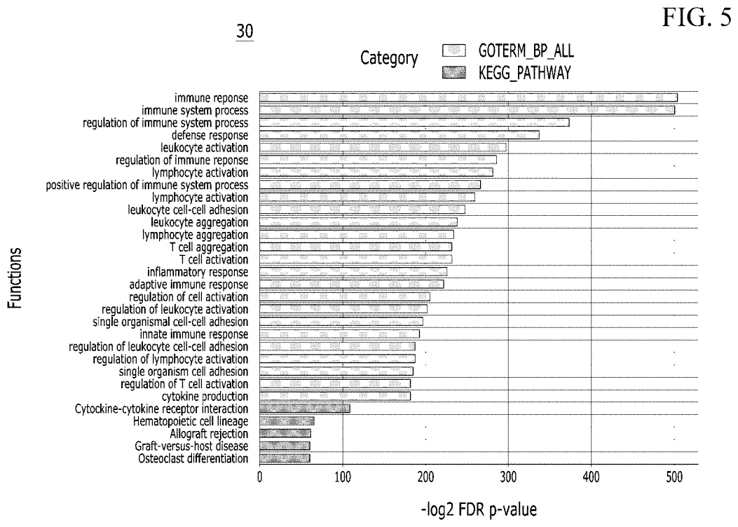

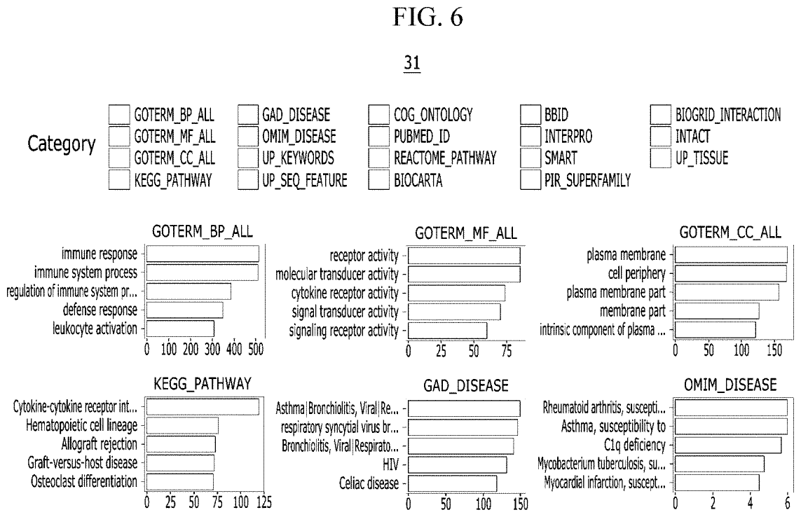

FIG. 5 and FIG. 6 are examples of enrichment analysis results for a gene module coded with a color name of black.

Specifically, FIG. 5 shows an example of enrichment analysis result 30 of a gene module coded with the color name of black. Here, the enrichment analysis of the gene module is performed for gene ontology and KEGG pathway. The term “category” means a database, and GOTERM_BP_ALL is a database of biological process term in gene ontology, and KEGG_PATHWAY is KEGG pathway database.

The enrichment analysis result 30 may be provided as a bar graph for biological and/or medical information that has a strong association with a gene module coded with the color name of black.

The enrichment analysis result 30 may be calculated as a false discovery rate (FDR) value. The gene module coded with the color name of black may be annotated as to have high relevance with immune response and immune system process of gene ontology, which are functions related to immunity Additionally, the gene module coded with the color name of black may be annotated as to be related with regulation of immune system process and defense response, and to be related to cytokine-cytokine receptor interaction, hematopoietic cell lineage, allograft rejection and the like of the KEGG pathway.

Referring to FIG. 6, the interpretation information generator 170 may provide an enrichment analysis result 31 of the gene module coded with the color name of black for various databases (categories) other than GOTERM_BP_ALL and KEGG_PATHWAY shown in FIG. 5.

As above-described, the interpretation information generator 170 provides a result indicating that the gene module coded with the color name of black is very significantly associated with the overall immune activities such as immune response, defense response of a cell, control of immune system, T cell activation, and the like, in the databases of gene ontology, KEGG pathway, and the like.

In fact, the gene module coded with the color name of black is a gene module where important genes responsible for human immune system are clustered. In addition, referring to FIG. 3, the gene module coded with the color name of black has high correlations with pathomics data CE_TIL_DEN and CS_TIL_DEN indicating immune cells (lymphoplasma) existing in the cancer epithelium and the cancer stroma region, respectively. Thus, it is confirmed that parameters (individual pathomics data) associated with immune cells in the pathomics data is related to gene modules with immunological features.

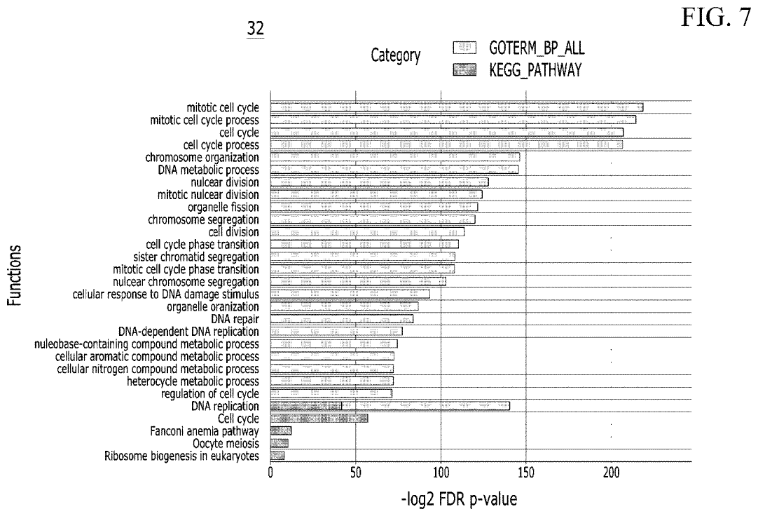

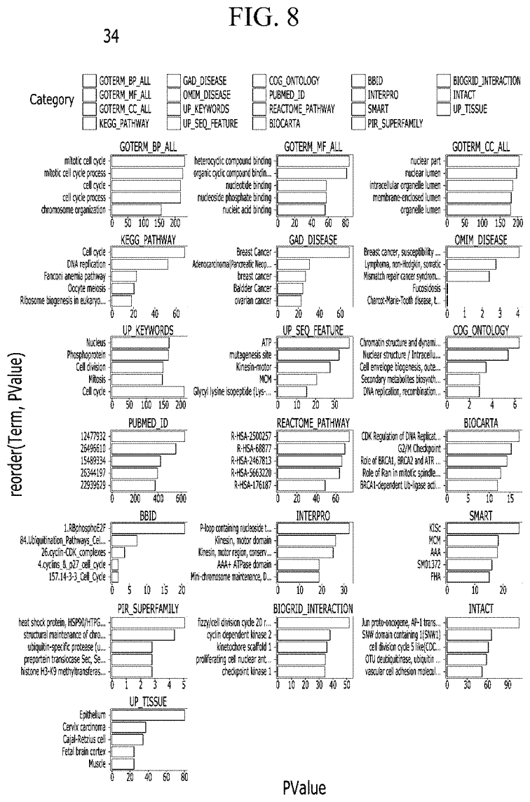

FIG. 7 and FIG. 8 are example diagrams showing enrichment analysis results for a gene module coded with a color name of yellow.

Specifically, FIG. 7 shows an example diagram of enrichment analysis result 32 of a gene module coded with a color name of yellow for gene ontology and KEGG pathway. The term “category” described in FIG. 7 means a database. Here, GOTERM_BP_ALL refers to a biological process term database, and KEGG_PATHWAY refers to KEGG pathway database.

The enrichment analysis results 32 may be provided as a bar graph of biological and/or medical information that has a strong association with the gene module coded with the color name of yellow.

The enrichment analysis result 32 may be calculated as a false discovery rate (FDR) value. The gene module coded with the color name of yellow can be annotated as to be associated with mitotic cell cycle, mitotic cell cycle process, cell cycle, cell cycle process, and DNA replication of gene ontology, and to be associated with DNA replication and cell cycle of KEGG pathway.

Referring to FIG. 8, the interpretation information generator 170 may provide an enrichment analysis result 34 of a gene module coded with a color name of black for various databases (categories) besides GOTERM_BP_ALL and KEGG_PATHWAY shown in FIG. 7.

As above-described, the interpretation information generator 170 provides a result that the gene module coded with the color name of yellow is very significantly related with cell division being the most important in cancer cells, such as cell division, cycle of cell division, cell nucleus division, and the like.

Actually, the gene module coded with the color name of yellow is a gene module where genes related to cell division are clustered. In addition, referring back to FIG. 3, it can be seen that the gene module coded with the color name of yellow has a high correlation with pathomics data CE_PER and CE_PC_PER indicating the area of the cancer epithelium. This indicates that the larger the area of cancer epithelial cells becomes, the more genes/transcripts that are biologically related to the division of cancer cells get expressed. Thus, it is confirmed that parameters related to an area of cancer cell (individual pathomics data) in the pathomics data are related to gene modules with a feature of cancer cell division.

Hereinafter, more specific description about the enrichment analysis result of the gene module coded with the color name of yellow and databases will be followed.

In biological process term of gene ontology, a cell cycle associated with a yellow gene module is a biological process belonging to a term “cellular process”. Besides the cell cycle, the term “cellular process” includes cell activation, cell adhesion molecule production, cell communication, cell cycle checkpoints, and the like. In cell cycle term, cell cycle processes, meiotic cell cycles, regulation of cell cycles, and the like exist, and further a subgroup of biological process term exists. As such, the biological meanings of the pathomics data such as distribution, properties, and density of cancer cells, and the like in pathological images may be explained through biological process terms.

In the KEGG Pathway, a cell cycle related to the yellow gene module belongs to cell growth and death subordinate to cellular processes. Thus, relationships between various information such as disease mechanism, cell metabolism, and the like and histologic components of the pathomics data may be explained.

In BIOCARTA, biocarta terms associated with the yellow gene module are CDK regulation of DNA replication, cell cycle: G2/M checkpoint, role of BRCA1, BRCA2, ATR in cancer susceptibility, and the like. DNA replication and cell cycles are repeated results in gene ontology and KEGG pathway. In that the genes BRCA1 and BRCA2 are considered to be very important in breast cancer and have correlations with the pathomics data obtained from extracting histologic components by using surgical biopsy data of breast cancer patients, the result is very meaningful for explaining cancer relevance to the genes BRCA1 and BRCA2.

In the genetic association database (GAD), the GAD term associated with the yellow gene module is breast-cancer. The pathomics data related to the yellow gene module are parameters generally belonged to cancer epithelium (mitosis, degenerated & necrotic tumor cell, macrophage, nuclear grade 3, ductal carcinoma in situ (DCIS), etc.). For the pathomics data obtained from extracting the histologic component by using surgical biopsy data of a breast cancer patient, the result is meaningful in that the very significant GAD term (p-value=1.54E-21) in the breast cancer is extracted.

In OMIM, the term associated with the yellow gene module is “Breast cancer, susceptibility to”. From this, it may be explained that the pathomics data obtained from extracting histologic components by using surgical biopsy data of breast cancer patients has significant relationship with a breast cancer.

UnitProt keywords related to the yellow gene module are cell cycle, nucleus, cell division, mitosis, and the like. Since those terms are associated with an area of cancer epithelium of breast cancer, it may be considered that the previously known knowledge is reproduced.

In UniProt tissue specificity, the term related to the yellow gene module is tissue corresponding to epithelium. Since the yellow gene module is highly associated with the area of cancer epithelium, extraction of tissues significantly associated with the epithelium is a very important result.

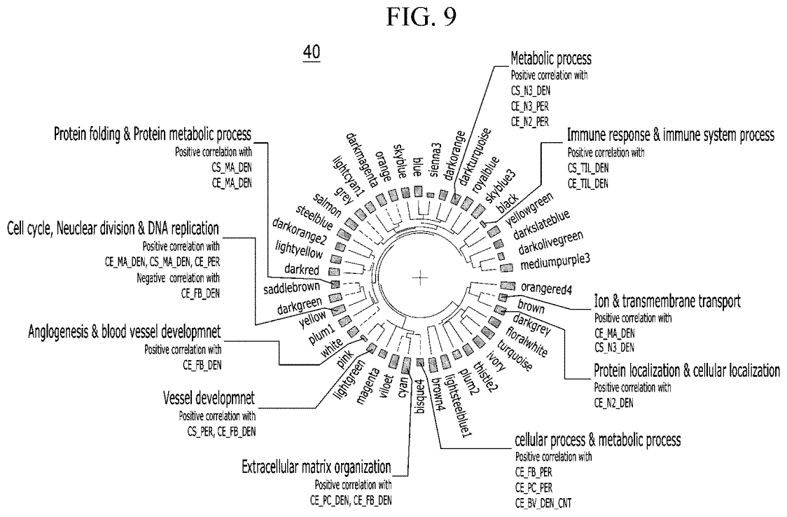

FIG. 9 is an example interface screen on which interpretation information is visually displayed, according to an embodiment.

Referring to FIG. 9, the interpretation information generator 170 may display a gene module associated with pathomics data of a patient and provide interpretation information annotated to the gene module, to the interface screen 40. The interpretation information may include functional information that is biological information, descriptive information that is medical information, and the like.

The interface screen 40 may display pathomics data on a gene module basis and display associated gene modules on pathomics data basis. In addition, the interpretation information generator 170 may hierarchically display the gene modules based on the hierarchical structure information among the gene modules to facilitate understanding of the interpretation information related to the pathomics data. The interface screen 40 may be obtained by assigning arbitrary colors to gene modules and visualizing as a circos plot through distance. The interface screen 40 visually describes the pathomics-gene module relationship having a significant correlation in FIG. 3. The interface screen 40 may provide pathomics data correlated with corresponding gene module along with the representative biological and/or medical information of each genetic module.

The interface screen 40 may display immune-related functions (immune response & immune system process) annotated to the gene module coded with the color name of black and further display information that the gene module has a positive relationship with individual pathomics data (CE_TIL_DEN, CS_TIL_DEN, etc.)

Therefore, it may be interpreted that the individual pathomics data (CE_TIL_DEN, CS_TIL_DEN, etc.) related to the number of lymphoplasma cells is associated with immune-related functions (immune response and immune system process). In addition, from a positive relationship, it may be inferred that the more lymphoplasma cells locates at cancer epithelial or cancer stroma in the slide image the more immunoreactivity activates. Such inference matches the relation of immune response between the number of pathologically interpretable lymphoplasma cells and biologically and/or medically interpretable cells. Thus, reliability of the analysis result of the AI pathology analyzer 10 may be evaluated based on the degree of match.

The interface screen 40 displays cell cycle, nuclear division, and DNA replication function that are annotated to the gene module coded with the color name of yellow. For example, information that there are positive relationships with CE_MA_DEN, CS_MA_DEN, CE_PER, and the like, and a negative relationship with CE_FB_DEN may be displayed together.

Therefore, patients with a large area of cancer in a slide image may be interpreted that the cancer cells are rapidly divided due to biologically fast cell cycle and have aggressive properties. Such an interpretation is consistent with a pathological interpretation, in that the rapid cancer cell division induces fast enlarging the size of a tumor and corresponding area of the slide image should be found to be large. Therefore, it may be verified that the size of pathologically interpretable tumor and the biological cell cycle are related features.

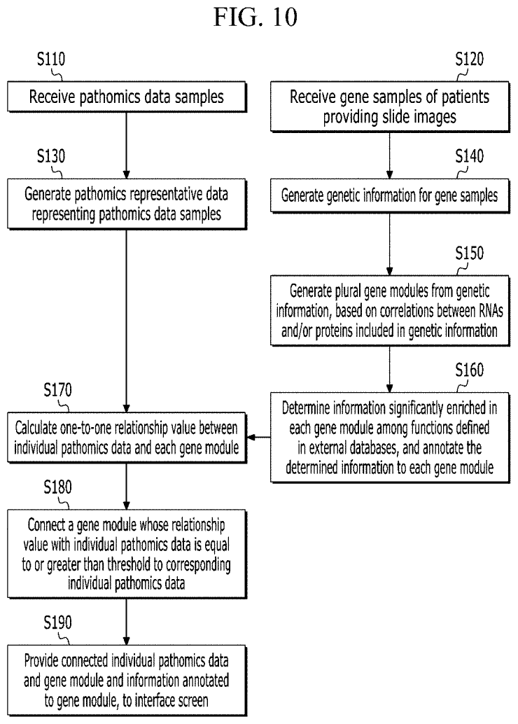

FIG. 10 is a flowchart showing a method for providing interpretation information of pathomics data according to an embodiment.

Referring to FIG. 10, an interpretation information providing system 100 receives pathomics data samples analyzed from slide images of patients (S110). The pathomics data samples includes quantitative data that is obtained by digitizing features of the slide images as the number of lymphoplasama cells located in the cancer epithelial and cancer stroma of the slide image, total amount of cancer epithelial and cancer stroma, and the like. The pathomics data samples may be raw data received from the AI pathology analyzer 10.

The interpretation information providing system 100 receives gene samples of the patients who provided the slide images (S120). Each gene sample may include RNA information and/or protein information, which are expression products of the gene, and include expression information of RNA and/or protein. The gene samples may include RNA expression data measured by transcriptomics techniques or protein expression data measured by proteomics techniques.

The interpretation information providing system 100 generates pathomics representative data representing the pathomics data samples (S130). The interpretation information providing system 100 calculates a representative value of individual pathomics data (p feature) constituting the pathomics data, by using the quantitative data included in the pathomics data samples. The interpretation information providing system 100 may determine a p-feature value representing K samples using, for example, a relative log cell-count (RLC) based data normalization technique.

The interpretation information providing system 100 generates genetic information from gene samples (S140). The interpretation information providing system 100 may calculate quantitative data of an individual gene (g gene) constituting the genetic information by using quantitative data included in the gene samples. The interpretation information providing system 100 may determine genetic information from K samples using, for example, a relative log-expression (RLE) based data normalization technique or a trimmed mean of M value based normalization technique.

The interpretation information providing system 100 generates a plurality of gene modules by grouping RNAs and/or proteins included in the genetic information 3, based on correlations thereamong (S150). The interpretation information providing system 100 may search a correlation network of data included in the genetic representative information by de-novo, or may analyze correlations based on unsupervised machine learning.

The interpretation information providing system 100 determines information significantly enriched in each gene module, from functions defined in external databases, and annotates the determined information to each gene module (S160). The external databases may include a biological database including gene feature information such as relationship information between biologically discovered genes and functions, pathways and interaction information, and the like, and medical databases utilized in medical fields such as biochemistry, medicine, pharmacy, and the like. The interpretation information providing system 100 may use gene set enrichment analysis (GSEA). The interpretation information providing system 100 may perform a significance test on association of functions extracted corresponding to each of the gene modules. The interpretation information providing system 100 may annotate significant enriched functions in each gene module as biological information, and may also annotate medical information related to the functions.

The interpretation information providing system 100 calculates a one-to-one relationship value (correlation value) between individual pathomics data included in the pathomics representative data and each gene module (S170). As shown in FIG. 3, the interpretation information providing system 100 may calculate a one-to-one relationship value between individual pathomics data and each gene module. The interpretation information providing system 100 may shorten the value of each gene module in a designated manner and then calculate a relationship with individual pathomics data.

The interpretation information providing system 100 connects a gene module whose relationship value with individual pathomics data is equal to or greater than a threshold to a corresponding individual pathomics data (S180). For example, the interpretation information providing system 100 may connect a gene module (color name of black) whose relationship values with the individual pathomics data CE_TIL_DEN and CS_TIL_DEN are greater than or equal to the threshold to CE_TIL_DEN and CS_TIL_DEN, respectively. Here, the gene module coded with the color name of black may be a gene module annotated with at least one function (for example, immune response and immune system process) and medical information related to the function.

The interpretation information providing system 100 provides the connected individual pathomics data and the gene module, and the annotated information to the gene module on the interface screen (S190). The annotated information may be used as interpretation information for individual pathomics data.

The order of processes shown in FIG. 10 may be changed according to a design, and the operations may be performed sequentially or in parallel.

FIG. 11 is a hardware configuration diagram of a computing device according to an embodiment.

Referring to FIG. 11, the interpretation information providing system 100 executes, in a computing device 300 operated by at least one processor, a program including instructions described to perform operations of the present disclosure. The program may be stored in a computer readable storage medium, and distributed as stored thereon.

The hardware of the computing device 300 may include at least one processor 310, a memory 330, a storage 350, and a communication interface 370, and may be connected via a bus. In addition, hardware such as an input device, an output device, and the like may be included. The computing device 300 may be equipped with a variety of software including an operating system executable the program.

The processor 310 is a device for controlling the operation of the computing device 300 and may be various types of processors for processing instructions included in a program. For example, the processor 310 may be a central processing unit (CPU), a micro processor unit (MPU), a micro controller unit (MCU), a graphic processing unit (GPU), and the like. The memory 330 loads the program such that the instructions described to perform the operations of the present disclosure are processed by the processor 310. The memory 330 may be, for example, a read only memory (ROM), a random access memory (RAM), and the like. The storage 350 stores various data, programs, and the like required to perform the operations of the present disclosure. The communication interface 370 may be a wired/wireless communication module.

The above-described embodiments of the present disclosure are not only implemented through an apparatus and a method, but may also be implemented through a program for embodying functions corresponding to the configuration of the embodiments of the present disclosure or a recording medium where the program is recorded.

While the present disclosure has been illustrated and descried with reference to embodiments thereof, the right scope of the present disclosure is not limited thereto. Further, it will be understood by a person of ordinary skill in the art that various changes in form and detail may be made therein without departing from the spirit and scope of the present disclosure as defined by the following claims.

What is claimed is:

1. An operation method of a computing device operated by at least one processor, the operation method comprising:
receiving pathomics data samples analyzed from slide images of patients and gene samples of the patients;
generating a plurality of gene modules by grouping genetic information included in the gene samples;
annotating information of databases significantly enriched in each of the gene modules, to a corresponding gene module;
based on one-to-one correlation values between the plurality of the gene modules and a plurality of individual pathomics data representing the pathomics data samples, extracting connectivity between the plurality of the individual pathomics data and the plurality of gene modules; and
connecting information annotated to each gene module and the individual pathomics data connected to the corresponding gene module.

2. The operation method of claim 1, wherein generating the plurality of gene modules comprises
based on correlations among RNAs and/or proteins included in the gene samples, modularizing the RNAs and/or proteins into the plurality of gene modules.

3. The operation method of claim 2, wherein each of the gene samples includes quantitative data that are obtained through measuring the RNAs and/or proteins by transcriptome analysis and/or proteome analysis.

4. The operation method of claim 1, wherein the databases are selected from databases that provide relationship information between biologically discovered genes and functions, gene feature information including pathways and interaction information, and medicine and pharmacy information.

5. The operation method of claim 1, wherein annotating information of databases comprises determining information of the databases significantly enriched in each of the gene modules through enrichment analysis.

6. The operation method of claim 1, wherein extracting the connectivity comprises shortening a value of each of the gene modules in a designated method and determining existence of a relationship between each of the gene modules and each individual pathomics data by using the shortened value of each of the gene modules.

7. The operation method of claim 1, further comprising providing information annotated to each of the gene modules as interpretation information of individual pathomics data connected to corresponding gene module.

8. The operation method of claim 1, wherein the individual pathomics data is a parameter representing cellular information and structural information of a pathological image, and
wherein a value of the individual pathomics data is determined by a representative value of the quantitative data of corresponding parameter in the pathomics data samples.

9. A computing device comprising:
a memory; and
at least one processor that executes instructions of a program loaded in the memory,
wherein the processor generates a plurality of gene modules by grouping genetic information of patients, determines a gene module correlated with pathomics data among the plurality of gene modules, and connects information of databases significantly enriched in each of the gene modules to the pathomics data correlated with corresponding gene module,
wherein the pathomics data is composed of parameters representing cellular information and structural information of pathological images and each parameter is represented as quantitative data, and
wherein the pathological images are obtained from the patients who provide the genetic information.

10. The computing device of claim 9, wherein the processor modularizes RNAs and/or proteins into the plurality of gene modules based on correlations among the RNAs and/or the proteins included in the genetic information.

11. The computing device of claim 9, wherein the processor determines information of the databases significantly enriched in each genetic module through enrichment analysis.

12. The computing device of claim 9, wherein the processor shortens a value of each of the gene modules in a designated method, calculates a correlation value between each of the gene module and individual pathomics data included in the pathomics data by using the shortened value of each gene module, and makes a relationship between the individual pathomics data and a gene module whose correlation value is equal to or greater than a threshold.

13. The computing device of claim 9, wherein the processor annotates information of databases significantly enriched in each of the gene modules to a corresponding gene module, and provides the information annotated to each of the gene modules as interpretation information of pathomics data connected to corresponding gene module.

14. A program stored on a non-transitory computer-readable storage medium, the program comprising instructions for causing a computing device to execute:
generating a plurality of gene modules by grouping genetic information of patients;
annotating information of databases significantly enriched in each gene module to a corresponding gene module;
determining a gene module correlated with pathomis data based on correlation values between the pathomics data and the plurality of genetic modules; and
storing connectivity between the plurality of the gene modules and the pathomics data extracted based on the correlation values, and the information annotated to each of the gene modules,
wherein the pathomics data is composed of parameters representing cellular information and structural information of pathological images, and each of the parameters is represented as quantitative data, and
wherein the pathological images are information obtained from the patients who provide the genetic information.

15. The program of claim 14, wherein annotating the information of databases comprises determining information of the databases significantly enriched in each of the gene modules through enrichment analysis, and annotating the information of the databases significantly enriched in each of the gene modules to a corresponding gene module.

16. The program of claim 14, further comprising instructions for causing a computing device to execute providing the information annotated to each of the gene modules as interpretation information of the pathomics data based on a connectivity between the pathomics data and the plurality of gene modules.
